(19) United States
(12) Patent Application Publication
(10) Pub. No.: US 2012/0071838 A1
(43) Pub. Date: Mar. 22, 2012

(54) RAPID EXCHANGE ASPIRATION CATHETERS WITH LUMENS CONFIGURED FOR OPTIMIZED FLOW

(75) Inventor: Shawn P. Fojtik, Park City, UT (US)

(73) Assignee: CONTROL MEDICAL TECHNOLOGY, LLC, Park City, UT (US)

(21) Appl. No.: 12/888,313

(22) Filed: Sep. 22, 2010

(51) Int. Cl. A61M 25/00; A61M 1/00; A61M 25/09

(52) U.S. Cl. 604/239; 604/523; 604/528

(57) ABSTRACT

A rapid exchange aspiration catheter includes a distal portion with a round cross-sectional shape, taken transverse to a longitudinal axis of the catheter. A primary lumen through the catheter has a size and a shape, and extends through the catheter at a location that are optimized for aspiration. In particular, the size of the primary lumen is maximized relative to the outer diameter of the catheter by minimizing the thicknesses of interior walls of the catheter. Such optimization maximizes the rate at which fluid flows through the aspiration catheter without compromising the ability of the catheter to withstand negative pressure (i.e., a vacuum). The present invention also includes systems in which the catheter is used in conjunction with a guide catheter and a wire, systems in which the catheter is used in conjunction with an aspiration element, and methods of using the catheter.

### TECHNICAL FIELD

The present invention relates generally to rapid exchange catheters and, more specifically, to rapid exchange catheters for aspirating objects and materials from the body of a subject. In particular, the present invention relates to rapid exchange catheters with distal portions that have round outer cross-sectional shapes, taken transverse to their longitudinal axes, primary lumens with round cross-sectional shapes, take transverse to their longitudinal axes, of optimized or maximized size, and smooth distal ends. In addition, the present invention relates to systems and methods for aspirating objects or materials from the body of a subject.

### BACKGROUND OF RELATED ART

So-called “over-the-wire” (OTW) catheters typically include a single, large lumen that runs the entire length of the catheter, from distal tip to proximal tail. OTW catheters have been used for a variety of purposes, including angiography; drainage; as “crossing catheters” that extend through chronic total occlusions (CTOs) in a subject's peripheral blood vessels, which includes blood vessels in the arms and legs; and as peripherally inserted central catheters (PICCs). When used to aspirate objects or material from the body of a subject, an OTW catheter may be used anywhere in the subject's body. Typically, however, use of OTW catheters is reserved for procedures outside of the heart.

A guide wire is typically used to facilitate the introduction of an OTW catheter into a subject's blood vessel. After the guide wire has been placed within the subject's blood vessel, a proximal end of a guide wire, which is the end located outside the subject's body, is inserted into the opening of the lumen of the OTW catheter at the distal end of the OTW catheter. The OTW catheter is then advanced distally into the blood vessel over the guide wire. When the OTW catheter is used to aspirate an object (e.g., a blood clot, such as a thrombus or an embolus, etc.) or material (e.g., tissue to be analyzed, etc.) from the body of a subject, advancement of the OTW catheter into the blood vessel may continue until the distal tip of the OTW catheter is located adjacent to or against the object (e.g., a blood clot, such as a thrombus or embolus, etc.) or material to be removed. The guide wire may then have to be removed so that an aspiration syringe may be coupled (e.g., with a luer lock or otherwise, as known in the art) to the proximal end of the catheter. With the aspiration syringe in place, a negative, or aspiration, pressure may be applied to the lumen of the OTW catheter, and the object or material at the distal end of the OTW catheter may be drawn into and through the lumen. During aspiration, the catheter may be advanced into or withdrawn from the object or material to be removed. The aspirant may be drained into a filter (e.g., a 20 μm to 150 μm filter, etc.) to enable examination of the object or material that has been removed from the subject's body with the catheter.

Since the guide wire must extend through the entire lumen of an OTW catheter, and since the proximal end of the guide wire must be held in place while the OTW catheter advances into the blood vessel, the guide wire must be at least twice as long as the OTW catheter, with at least half of the length of the guide wire remaining outside of the subject's body. The undesirable consequences of such long guide wires (e.g., the effort required to thread a catheter over the guide wire, the potential for contaminating the guide wire by dropping it on the floor, the expenses associated with long guide wires, etc.) led to the development of so-called “rapid exchange” or “RX” catheters, which require that only a small length of a guide wire (e.g., about 20 cm to about 50 cm, etc.) be present outside of a subject's body to enable introduction of an RX catheter into the subject's body.

The construction of a rapid exchange catheter 110, an example of which is shown in FIG. 1, typically includes a primary element 120 and an RX element 140. The primary element 120 extends the entire length of the catheter 110. In the depicted example, the primary element 120 includes a primary lumen 122 that extends along the entire length of the catheter 110. The RX element 140 is located only adjacent to the distal end 114 of the catheter 110 (e.g., along about 15 cm to about 30 cm of a distal portion 115 of the catheter 110). An RX lumen 142, which extends through the RX element 140, is dedicated for use with a stylet or guide wire, and typically has a smaller inner diameter (ID) than the primary lumen 122 of the primary element 120 of the catheter 110.

In the depicted example, the distal portion 115 of the catheter 110 has the appearance of two fused tubes, with elongate indentations 117 in the outer surface 116 of the catheter 110, along both sides of the catheter 110 at a junction between the primary element 120 and the RX element 140. When the catheter 110 is used with a guide catheter, the elongate indentations 117 create undesirable gaps or voids between the inner surface 212 of the guide catheter 210 and the outer surface 116 of catheter 110, as shown in FIG. 2. Gaps between a catheter 110 and a guide catheter 210 are particularly problematic in situations where size constraints are placed upon the useful size of the guide catheter 210, such as the 6 French (0.078 inch or 2 mm outer diameter (OD)) limitation on guide catheters used to facilitate the entry of aspiration catheters into coronary arteries. The lumen of such a catheter typically has an ID of about 0.069 inch to about 0.071 inch. Limitations on the ID of a guide catheter translate to limitations on the outer diameters (ODs) and lumen IDs of the catheters that may be used in such a guide catheter.

Another example of an RX catheter 110′ is illustrated by FIG. Like the RX catheter 110 shown in FIGS. 1 and 2, RX catheter 110′ includes a primary element 120′ that extends along the entire length of the catheter 110′ and an RX element 140′ located adjacent to a distal end (not shown) of the catheter 110′. Unlike the RX catheter 110 shown in FIGS. 1 and 2, the outer surface 116 of the distal portion of RX catheter 110′, which includes both the primary element 120′ and the RX element 140′, is smooth, lacking indentations, as shown by FIG. 3. Nonetheless, a portion 125′ of the wall of RX catheter 110′ protrudes into the primary lumen 122′ of the RX catheter 110′, which may undesirable consume valuable area within the primary lumen 122′ and, thus, reduce fluid flow through the primary lumen 122′, create a structure that may obstruct the flow of particles through the primary lumen 122′, or both.

Once a catheter (an OTW catheter, and RX catheter, etc.) has been introduced into the body of a subject, the guide wire may be left in place. The coupling of other elements (e.g., syringes, etc.) to the proximal end of the catheter may be enabled by connecting a Y-adapter or a T-adapter to the proximal end of the catheter, with the guide wire extending through one of the arms of the adapter. The guide wire may be secured in place relative to the adapter and the length of the catheter by known means (e.g., a screw associated with the arm of the adapter through which the wire extends, etc.) and another element (e.g., a syringe, etc) may communicate with the catheter through the other arm of the adapter.

### SUMMARY

A catheter that incorporates teachings of the present invention may be configured for aspirating substances from the body of a subject. In various embodiments, an aspiration catheter of the present invention includes features that facilitate rapid exchange with a guide wire. A primary lumen of the catheter may have a smooth inner surface and an optimized cross sectional area relative to the outer cross sectional area of the catheter. A catheter of the present invention may have a smooth distal end.

A catheter of the present invention may have a smooth outer surface along a distal portion of its length. The smooth outer surface may be provided by a round cross-sectional outer shape, taken transverse to the longitudinal axis of the syringe. The smooth outer surface imparts the catheter with the appearance of a tube along the distal portion its length. As used herein, the term “round” includes circular, ovoid and other shapes that generally lack corners, sharp bends or inwardly or outwardly protruding features.

The outer surface and other features of the catheter are defined by a wall of the catheter, which is also referred to herein as a “catheter wall.” Along one side of the length of the catheter, the catheter wall is thicker than a region of the catheter wall that extends along the opposite side of the catheter. These diametric regions are respectively referred to as a “thick region” and a “thin region.”

A primary lumen of the catheter, which is defined by the catheter wall, also has a round cross-sectional shape, taken transverse to the longitudinal axis of the primary lumen, and a smooth interior surface along substantially the entire length of the catheter. Optimization of the cross sectional area of the primary lumen may be achieved by way of the diametric, longitudinally extending thick and thin regions of the catheter wall. This arrangement may result from the use of slightly different cross sectional shapes for the outer periphery of the catheter and the inner periphery of the primary lumen, from slightly offsetting the axes that run along the lengths, or longitudinal axes, of the catheter and its primary lumen, or from any other suitable design. The smooth interior surface of the primary lumen and its optimized size, relative to the outer dimensions of the catheter, are tailored to optimize the flow of fluids through the catheter.

In addition to the primary lumen, a catheter that incorporates teachings of the present invention also includes at least one secondary lumen extending through the thick region of the catheter wall. The at least one secondary lumen may extend along a portion of the length of the catheter, along substantially the entire length of the catheter, or along the entire length of the catheter. In specific embodiments, the at least one secondary lumen extends through the thick region of the catheter wall at the distal portion of the catheter, with a proximal end opening to the outer surface of the catheter and the opposite distal end opening a distal end of the catheter. In some embodiments, a catheter of the present invention may include one or more additional secondary lumens that extend through the thick region of the catheter wall along substantially the entire length or through the entire length of the catheter.

A distal end of a catheter of the present invention may be smooth. In some embodiments, a distal tip of the catheter may include a rounded, smoothed feature, which may be configured to scoop loose or soft tissues or materials without injuring the subject.

In another aspect, the present invention includes systems for aspirating substances from the body of a subject. One embodiment of such a system includes the aspiration catheter, a wire (e.g., a guide wire, etc.), and a guide catheter. The outer surface of the aspiration catheter (including its OD) and the inner surface of the guide catheter (including its ID) are configured to enable introduction of the aspiration catheter into the lumen of the guide catheter; i.e., the OD of the aspiration catheter is slightly smaller than the ID of the guide catheter. The outer surface of the aspiration catheter is configured to eliminate any unnecessary gaps between the outer surface of the aspiration catheter and the inner surface of the guide catheter as the aspiration catheter is present within the guide catheter, while maximizing the cross-sectional area of the primary lumen through the aspiration catheter.

In some embodiments, an aspiration system of the present invention may include a catheter, a wire, an optional guide catheter, and an aspiration syringe. In a specific embodiment, the aspiration syringe may comprise a syringe of the type disclosed by U.S. Pat. No. 7,534,234, the aspiration syringe described by U.S. patent application Ser. No. 12/723,610, filed on Mar. 12, 2010, or the aspiration elements of the multi-barrel syringe described by U.S. Pat. No. 7,674,247, the disclosures of each of which are hereby incorporated herein, in their entireties, by this reference. An aspiration system of the present invention may also include other elements that facilitate the removal of tissues or other substances from the body of a subject.

The present invention also includes various methods, including methods for using aspiration catheters. An aspiration catheter may be inserted into the body of a subject along a guide wire that has been positioned in a manner known in the art. In such a method, a proximal end of the guide wire may be introduced into the distal end of a secondary lumen that opens to the distal end of the catheter, and pushed through the secondary lumen until it emerges from the proximal end of the secondary lumen. While holding the proximal end of the guide wire in place outside of the body of the subject, the catheter may be introduced into the body of the subject by moving the catheter distally along the guide wire. The limited length of the secondary lumen provides the catheter which rapid exchange capability, which eliminates the need for a guide wire that has more than twice the length of the catheter. The smooth distal end of the catheter enables introduction of the catheter into the subject's body while minimizing or eliminating the potential for injury to the subject's body.

With the catheter in place in the subject's body, various objects (e.g., a blood clot, such as a thrombus or embolus, etc.) or materials may be aspirated from the subject's body. In situations where the object or material adheres to the body of the subject (e.g., to the interior surface of a blood vessel, etc.), the distal end of the guide wire may be withdrawn into the secondary lumen of the catheter, then the catheter may be nudged forward (distally), forcing its smooth distal end into the object or material to scoop and loosen the same without injuring the subject. Embodiments in which the distal tip of the catheter is rounded are particularly useful for this purpose.

Other aspects, as well as features and advantages of various aspects, of the present invention will become apparent to those of ordinary skill in the art through consideration of the ensuing description, the accompanying drawings and the appended claims.

### BRIEF DESCRIPTION OF THE DRAWINGS

In the drawings:

FIGS. 1 and 2 illustrate an example of a prior art aspiration catheter;

FIG. 3 depicts another example of a prior art aspiration catheter;

FIG. 4 is a perspective view of a distal portion of a catheter that incorporates teachings of the present invention;

FIG. 5 is a cross-sectional representation of an embodiment of the catheter shown in FIG. 4;

FIG. 6 is a cross-sectional representation of another embodiment of the catheter shown in FIG. 4;

FIG. 7 is a cross-sectional representation taken along the length of a distal portion of the catheter shown in FIG. 4;

FIG. 8 is a cross-sectional representation taken along a portion of the length of an embodiment of the catheter shown in FIGS. 4 and 7;

FIG. 9 is a cross-sectional representation taken transverse to the length of an embodiment of the catheter shown in FIGS. 4, 7 and 8, at a location proximal to the distal portion;

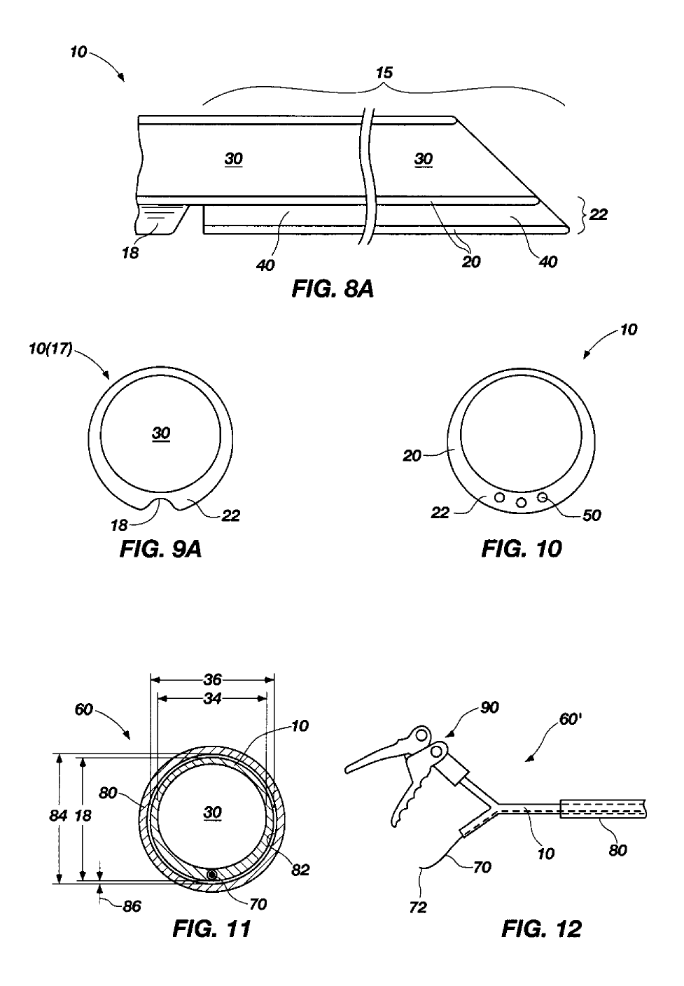

FIG. 8A is a cross-sectional representation taken transverse to another embodiment of the catheter shown in FIGS. 4 and 7, at a location proximal to the distal portion of the catheter;

FIG. 9A is a side view of the catheter shown in FIG. 8A;

FIG. 10 is a cross-sectional representation of an embodiment of catheter that includes a plurality of lumens extending through a longitudinally extending thickened region of the wall of the catheter;

FIG. 11 is a cross-sectional representation of an embodiment of a system that includes a catheter of the present invention; and

FIG. 12 schematically depicts an aspiration system that includes a catheter of the present invention.

### DETAILED DESCRIPTION

According to Poiseuille's law, the laminar flow rate (Q) of fluid through the opening of a tube, such as the lumen of a catheter, is affected by a number of factors, including the diameter working lumen's radius (r), the pressure differential between one end of the lumen and the other (P), the length of the tube (L), and the viscosity of the fluid (η). Poiseuille's law is exemplified by the following formula:

$$Q = \frac{π {r}^{4} P}{8 η L}$$

From the formula, particularly from the fact that when the size of the opening (r) is considered, it is increased to the fourth power, it is apparent that an increase in the radius (r) (or cross-sectional area) of the tube has a more significant impact on the laminar rate (Q) at which fluid may flow through the tube than a proportionate increase (or even a much greater increase) than any of the other factors that are considered by Poiseuille's law. For example, when the only change to a catheter is a ten percent (10%) increase in the radius of its lumen, the laminar flow rate (Q) of that catheter increases by over 45% (1.1×1.1×1.1×1.1=1.4641). Increases in the rates at which fluids flow through catheters are particular desirable where the catheters are used to aspirate or drain material from the body of a subject.

The present invention includes RX catheters with primary lumens that are shaped and configured for optimal fluid flow rates. With reference to FIG. 4, a catheter 10 that embodies teachings of the present invention is an elongate element with a proximal end (not shown) and an opposite distal end 14. The proximal end is configured to be located outside of a subject (or patient), and is the end by which a healthcare provider accesses the catheter once it has been positioned within the subject's body. The distal end 14 is configured to be placed at a desired location within the body of the subject.

The catheter 10 and its various features are defined by a catheter wall 20. An outer surface 16 of the catheter wall 20 and, thus, of the catheter 10 may be smooth. A rapid exchange portion of the catheter 10, which is referred to as a “distal portion” 15, has a round cross-sectional shape along substantially its entire length, except for the proximal-most and distal-most parts of the distal portion 15. The specific outer cross-sectional shape of a catheter 10 of the present invention may, in some embodiments, be the same as the cross-sectional shape of a guide catheter (see FIG. 11) through which the catheter 10 is configured to be introduced into the body of a subject.

The catheter wall 20 defines a primary lumen 30 through the entire length or through substantially the entire length of the catheter 10. The primary lumen 30 has a smooth surface, a generally round or rounded cross-sectional shape (taken transverse to its length), and lacks inwardly protruding features.

In various embodiments, the catheter 10 and its primary lumen 30 are shaped and positioned in a way that imparts the catheter wall 20, at least along the distal portion 15 of the catheter 10, with a longitudinally extending thick region 22 and a longitudinally extending thin region 24. The thick region 22 and the thin region 24 may be diametrically positioned.

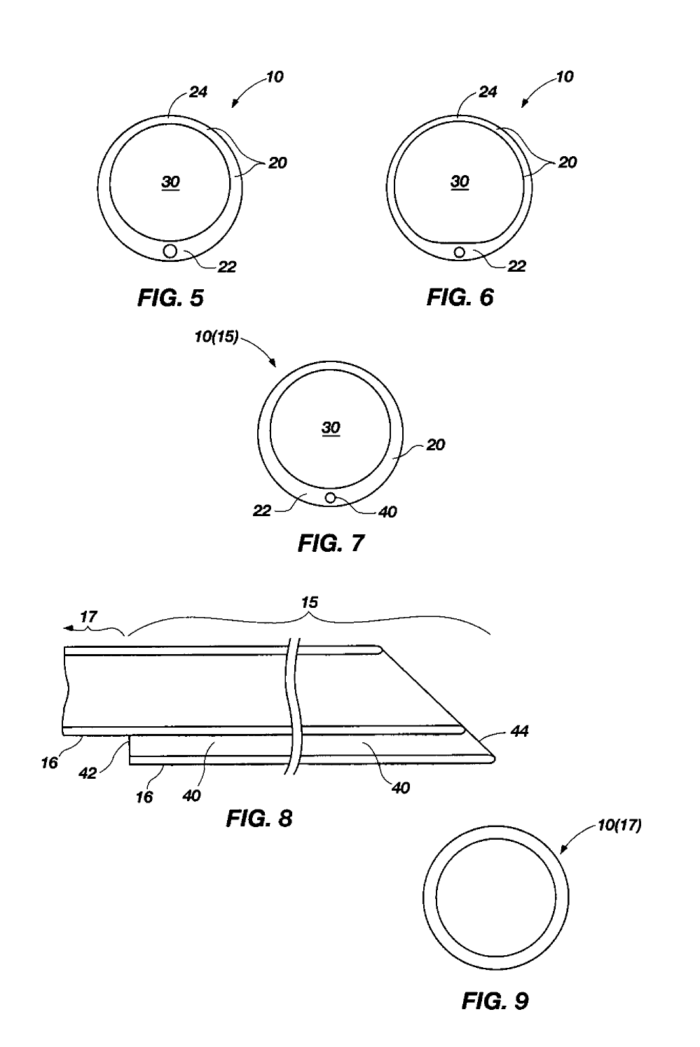

In embodiments such as that illustrated by FIG. 5, the outer cross-sectional shape of the catheter 10 and the cross-sectional shape of the primary lumen 30 of the catheter 10 are substantially the same, but the primary lumen 30 is not situated concentrically within the catheter 10. Stated another way, the primary lumen does not share a longitudinal axis with the entire catheter 10.

In some embodiments, like that depicted by FIG. 6, the thick region 22 and thin region 24 of the catheter wall 20 may be the result of the use of different cross-sectional shapes for the primary lumen 30 and the outer surface 16 of the catheter 10. Of course, the position of the primary lumen 30 within the remainder of the catheter 10 also determines the locations and thicknesses of the thick and thin regions 22 and 24.

These and similar embodiments may facilitate minimization of the thicknesses of interior walls of the catheter 10. In some embodiments, such minimization includes designing the catheter 20 to remove of about 0.005 inch, or about 0.0127 mm, relative to the thickness of the internal walls of an existing RX catheter. The thin region 24 of the catheter wall 20, as well as the remainder of the catheter wall 20, may be thick enough to withstand pressures of up to about 760 mm Hg. In specific embodiments, the thin region 24 of the catheter wall 20 may have a thickness of only about 0.005 inch, or about 0.13 mm. Such an embodiment may include a catheter wall 20 with a thick region 22 that measures about 0.020 inch or less, or about 0.51 mm or less, across. From top to bottom, the cumulative wall thickness of a catheter of the present invention may be only about 0.025 inch, or about 0.64 mm. In contrast, the total thickness of the walls from top to the bottom of existing RX catheters, including the IDs of their guide wire lumens, may measure about 0.030 inch or more, or about 0.76 mm or more.

With returned reference to FIG. 4, and with additional reference to FIGS. 7, 8 and 9, a secondary lumen 40 is located in a distal portion 15 of the catheter 10. More specifically, the secondary lumen 40 extends through the thick region 22 of the catheter wall 20 in the distal portion 15 of the catheter 10. The secondary lumen 40 provides the catheter 10 with its rapid exchange functionality and, therefore, has dimensions (e.g., a diameter of 0.016 inch, a slightly greater diameter than the diameter of the wire, etc.) that enable the secondary lumen 40 to receive a wire 70 (see FIGS. 11 and 12) (e.g., a guide wire, etc.), and that enable the wire 70 to slide through the secondary lumen 40.

In the illustrated embodiment (see FIG. 8), the secondary lumen 40 includes a distal end 44 that opens to the distal end 14 of the catheter 10. An opposite proximal end 42 of the secondary lumen 40 opens to the outer surface 16 of the catheter 10, at a location where the OD of the catheter decreases. When the catheter 10 is used with a wire 70 and a guide catheter 80 (FIGS. 11 and 12), the decreased OD of the more proximal portions of the catheter 10 provides a space between the outer surface 16 of the catheter 10 and the surface 84 of the lumen 82 of the guide catheter. The dimensions of that space accommodate the thickness of the wire 70.

Various embodiments of the catheter 10 include secondary lumens 40 with lengths of about 5 cm to about 50 cm. In other embodiments, the length of the secondary lumen 40 of a catheter 10 may be about 10 cm to about 30 cm. Of course, catheters 10 with secondary lumens 40 of other lengths are also within the scope of the present invention.

As shown in FIGS. 8 and 9, the OD of the catheter 10 diminishes on the proximal side 17 of the distal portion 15. In tapers at the proximal end of the distal

In other embodiments, such as that shown in FIGS. 8A and 9A, the thick region 22 of the catheter wall 20 extends substantially the entire length of the catheter 10. In such an embodiment, the thick region 22 carries the secondary lumen 40 at the distal portion 15 of the catheter 10, while in more proximal portions 17 of the catheter 10, the thick region 22 carries an elongate indentation 18. The elongate extension 18 may accommodate a wire 70 (see FIG. 11) as the catheter 10 is introduced into the body of a subject. In embodiments where the catheter 10 is configured for introduction into the body of a subject through a guide catheter 80 (see FIG. 11), the elongate extension 17 may enable optimization of the outer dimensions of the catheter 10 and, thus, of the primary lumen 30 extending through the catheter 10.

As best seen in FIG. 4, the distal end 14 of the catheter 10 may be oriented at an angle of about 15° to about 90° to the longitudinal axis of the catheter. In some embodiments, the angle at which the distal end 14 is oriented relative to the length of the catheter 10 may be about 30° to about 45°.

The distal end 14 of the catheter 10 lacks any protruding features. Rather, the entire distal end 14, including the location where the distal end 44 of the secondary lumen 40 opens to the distal end 14, is smooth. Thus, the distal end 14 of the catheter 10 lacks any protruding features. In various embodiments, some or all of the corners at the distal end 14 may be rounded or otherwise smoothed to enable smooth movement of the catheter 10 through vessels within the body of a subject.

In some embodiments, the distal end 14 of the catheter 10 may have a rounded shape. Such a shape may result from the use of a straight, angled cut (i.e., at an acute angle) to form the distal end of the catheter 10. The smooth, rounded shape of the distal end 14 of such an embodiment of catheter 10 may be useful for scooping and, thus, removing tissues (e.g., embolisms, etc.) or materials from within a vessel or other feature of the body of a subject.

As shown in FIG. 10, a catheter 10 of the present invention may include one or more additional lumens 50 extending through the thick region 22 of the catheter wall 20. These additional lumens 50 may extend partially along the length of the catheter 10, substantially the entire length of the catheter 10 (e.g., opening to the outer surface 16 of the catheter 10 just short of one or both its proximal end 12 and distal end 14, etc.), or the entire length of the catheter 10.

Turning now to FIGS. 11 and 12, an embodiment of an aspiration system 60 is shown that includes a catheter 10 of the present invention. In addition to the catheter 10, the aspiration system 60 includes a wire 70 (e.g., a guide wire, etc.) and a guide catheter 80. In embodiments where the wire 70 comprises a guide wire, the wire 70 may extend distally further into the body of a subject (e.g., about 10 cm further, about 15 cm further, about 20 cm further, about 25 cm further, etc.) than the guide catheter 80; i.e., a distal portion of the wire 70 may extend beyond the distal end 14 (FIG. 4) of the catheter 10.

Without limiting the scope of the present invention, the catheter 10 may comprise an angioplasty catheter, an aspiration catheter, a support catheter, or a crossing catheter, or any other catheter that is configured for use with a wire, such as a guide wire.

The catheter 10 has an OD 18 that is only slightly smaller than an ID 84 of a lumen 82 that extends through the length of the guide catheter 80. The difference, or clearance 86, between the OD 18 and the ID 84 is only large enough to enable the catheter 10 to slide through the lumen 82. In some embodiments, the clearance 86 is only large enough to receive a suitable lubricant, such as a sterile saline solution. By minimizing the clearance 86 between the ID 84 of the lumen 82 and the OD 18 of the catheter 10, the OD 18 of the catheter 10, as well as the cross-sectional area of its primary lumen 30, may be maximized, or optimized.

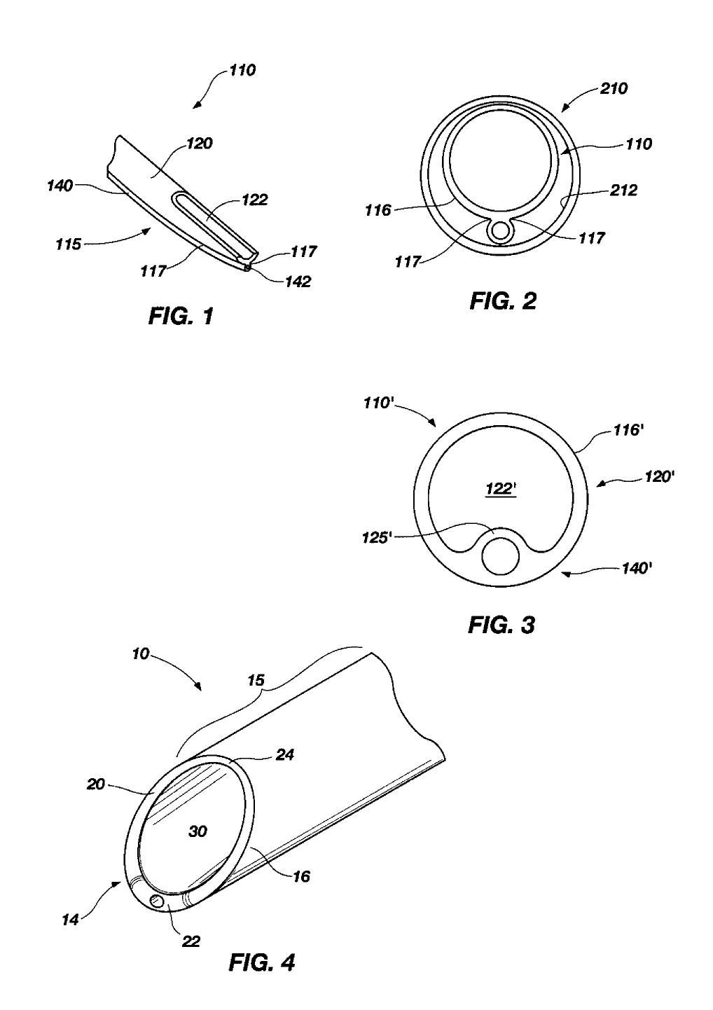

In a specific embodiment of such a system, a 6 French (F) catheter (i.e., a catheter with an OD of 2 mm, or 0.079 inch) is employed as the guide catheter 80. The lumen 82 of a 6 F guide catheter has an ID 84 of about 0.068 inch to about 0.072 inch. Thus, the OD 18 of the catheter 10 that may be used within such a guide catheter is less than ID 84. For example, the OD 18 of the distal portion 15 (FIG. 8) of the catheter 10 may be about 0.068 inch, while more proximal portions 17 (FIG. 8) of the catheter have an OD of about 0.054 inch. The primary lumen 30 of such a catheter 10 may have an ID 34 of more than 0.040 inch, to as much as about 0.045 inch, or more than 1.06 mm to about 1.14 mm, which represents more than a 10% increase over the 0.037 inch, or 1.02 mm, maximum diameter of the primary lumen of an existing RX catheter with a 0.068 inch OD 36 and smooth outer surface (FIG. 2), as well as a significant increase over the cross-sectional area of the primary lumen of an RX catheter with the appearance of two fused tubes (FIG. 1). Of course, similar, even more pronounced, differences are present in catheters with smaller ODs 36 (e.g., 0.065 inch, etc.).

As noted above, since a catheter 10 of the present invention has a diameter that is at least 10% greater than the diameter of an existing RX aspiration catheter with similar outer dimensions, the rate at which a particular fluid flows through the primary lumen 30 of a catheter 10 of the present invention is more than 45% greater than the rate at which that fluid flows, under the same pressure, through the existing RX aspiration catheter. In addition, the increased size of the primary lumen 30, as well as its smooth surfaces and lack of protruding features, enable it to accommodate larger particles (e.g., embolisms, other tissues, other substances, etc.) than the primary lumens of existing RX aspiration catheters.

While the clearance 86 between the OD 18 of the catheter 10 and the ID 84 of the lumen 82 is minimized, the longitudinally extending elongate indentation 17 in the catheter 10 and the secondary lumen 40 may accommodate the wire 70 while the catheter 10 is introduced into or removed from the lumen 82 of the guide catheter 80.

As depicted by FIG. 12, a system 60′ of the present invention may be used to aspirate substances (e.g., tissue or other materials) from the body of a subject. In addition to a catheter 10, a wire 70, and an optional guide catheter 80, such a system may include an aspiration element 90, such as a syringe, an automated aspiration device, or any other device that can communicate with the primary lumen 30 of the catheter 10 to generate a negative pressure (i.e., vacuum) within the primary lumen 30. Without limiting the scope of the present invention, a manually operated aspiration syringe, such as that described by U.S. Pat. No. 7,534,234 or the aspiration element of the multi-barrel syringe described by U.S. Pat. No. 7,674,247, may serve as the aspiration element 90 of a system 60′ of the present invention. An aspiration element 90, such as the aspiration syringe described by U.S. patent application Ser. No. 12/723,610, may also be configured to expel gases that escape from aspirated fluids held under negative pressure

Returning reference to FIGS. 8, 11 and 12, a catheter 10 of the present invention may be inserted into the body of a subject along a guide wire (an embodiment of wire 70) that has been positioned within the body of the subject in a manner known in the art. A proximal end 72 of the wire 70 may be introduced into the distal end 44 of the secondary lumen 40 that opens to the distal end 14 of the catheter 10. The proximal end 72 of the wire 70 may be pushed through the secondary lumen 40 until it emerges from the proximal end 42 of the secondary lumen 40 and, thus, from the distal portion 15 of the catheter 10. While holding the proximal end 72 of the wire 70 in place outside of the body of the subject, the catheter 10 may be introduced into the body of the subject by moving the catheter 10 distally along the wire 70. The limited length of the secondary lumen 40 provides the catheter 10 which rapid exchange capability, which eliminates the need for a wire 70 that has more than twice the length of the catheter. The smooth distal end 14 of the catheter 10 enables introduction of the catheter into the subject's body while minimizing or eliminating the potential for injury to the subject's body. Alternatively, or in addition, the catheter 10 may be introduced into the body of the subject using a guide catheter 80.

With the catheter in place in the subject's body, various tissues (e.g., a blood clot, such as a thrombus or embolus, etc.) and other substances may be removed from the subject's body. In situations where the object or material adheres to the body of the subject (e.g., to the interior surface of a blood vessel, etc.), the distal end of the guide wire may be withdrawn into the secondary lumen of the catheter, then the catheter may be nudged forward (distally), forcing its smooth distal end into the object or material to scoop and loosen the same without injuring the subject. Embodiments in which the distal tip of the catheter is rounded are particularly useful for this purpose.

Examples of procedures in which a catheter of the present invention may be used include, but are not limited to, general drainage; plural effusions; cyst aspiration; endometrial vacuum, suction and extraction; abscess, nephrostomy, and biliary drainage; lavage; wound evacuation; bone biopsy, spinal tap, and marrow extraction; liposuction; thrombus, embolectomy, and atherectomy; crossing, support, and distal access; embolic delivery; and other situations with fluids, gases, plasmas, and solids need to be removed.

Although the foregoing description contains many specifics, these should not be construed as limiting the scope of the invention or of any of the appended claims, but merely as providing information pertinent to some specific embodiments that may fall within the scopes of the invention and the appended claims. Other embodiments of the invention may also be devised which lie within the scopes of the invention and the appended claims. Features from different embodiments may be employed in combination. The scope of the invention is, therefore, indicated and limited only by the appended claims and their legal equivalents. All additions, deletions and modifications to the invention, as disclosed herein, that fall within the meaning and scopes of the claims are to be embraced thereby.

What is claimed:

1. A catheter, comprising:
a catheter wall with an outer surface and an inner surface, the inner surface defining a primary lumen of the catheter;
a proximal end; and
a smooth distal end, the inner and outer surfaces of the catheter wall having round cross-sectional shapes at a distal portion of the catheter wall, the distal portion of the catheter wall defining at least one secondary lumen of the catheter, with the at least one secondary lumen opening to the smooth distal end.

2. The catheter of claim 1, wherein a portion of the catheter wall through which the secondary lumen extends is thicker than a diametrical portion of the catheter wall.

3. The catheter of claim 1, wherein a length of the at least one secondary lumen is internally confined within the catheter wall.

4. The catheter of claim 1, wherein the secondary lumen extends only through the distal portion of the of the catheter wall, a proximal end of the secondary lumen opening to the outer surface of the catheter wall.

5. The catheter of claim 4, wherein the secondary lumen is configured to receive a guide wire and imparts the catheter with rapid exchange functionality.

6. The catheter of claim 5, wherein the secondary lumen extends proximally about 5 cm to about 50 cm from the smooth distal end.

7. The catheter of claim 1, comprising a plurality of secondary lumens.

8. The catheter of claim 7, wherein at least one secondary lumen of the plurality of secondary lumens extends through substantially an entire length of the catheter wall.

9. The catheter of claim 1, wherein at least one edge of the catheter wall that defines a boundary of the smooth distal end is rounded.

10. The catheter of claim 1, wherein the primary lumen opens through the smooth distal end.

11. The catheter of claim 1, wherein the smooth distal end is oriented at an angle to a longitudinal axis of the catheter.

12. The catheter of claim 8, wherein the smooth distal end is oriented at an angle of about 15° to about 90° to the longitudinal axis of the catheter.

13. The catheter of claim 8, wherein the smooth distal end is oriented at an angle of about 30° to about 45° to the longitudinal axis of the catheter.

14. The catheter of claim 1, wherein the catheter wall is configured to withstand negative pressure within the primary lumen of up to about 760 mm Hg.

15. An aspiration system, comprising:
a guide catheter with a lumen extending therethrough;
an aspiration catheter, with:
a catheter wall with an outer surface and an inner surface, the outer surface imparting the aspiration catheter with maximum possible dimensions for enabling the aspiration catheter to move through the lumen of the guide catheter, the inner surface defining a primary lumen of the aspiration catheter;
a proximal end; and
a smooth distal end, the inner and outer surfaces of the catheter wall having round cross-sectional shapes at a distal portion of the catheter wall, the distal portion of the catheter wall defining at least one secondary lumen of the aspiration catheter, with the at least one secondary lumen opening to the smooth distal end; and
a wire configured to extend through the at least one secondary lumen.

16. The system of claim 15, wherein the wire comprises a guide wire.

17. The system of claim 15, further comprising:
an aspiration element.

18. The system of claim 17, wherein the aspiration element comprises a hand-held, manually operable aspiration syringe.

19. The system of claim 15, wherein the guide catheter is a 6 French catheter, the aspiration catheter has an outer diameter of about 0.070 inch, and the primary lumen of the aspiration catheter has an inner diameter of at least about 0.040 inch.

20. The system of claim 19, wherein the primary lumen of the aspiration catheter has an inner diameter of at least about 0.045 inch.
